(19) United States
(12) Patent Application Publication
(10) Pub. No.: US 2025/0367363 A1
(43) Pub. Date: Dec. 4, 2025

(54) METHODS OF LOCAL IRRIGATION FOR TREATING INFECTIONS

(71) Applicant: Osteal Therapeutics, Inc., Dallas, TX (US)

(72) Inventors: Brian C. DE BEAUBIEN, Fenton, MI (US); Kevin WARNER, Dallas, TX (US); Jude PAGANELLI, Coronado, CA (US); William C. PUTNAM, Frisco, TX (US); Daniel PFLASTER, Charlotte, VT (US)

(73) Assignee: Osteal Therapeutics, Inc., Dallas, TX (US)

(21) Appl. No.: 19/299,036

(22) Filed: Aug. 13, 2025

Related U.S. Application Data
(63) Continuation-in-part of application No. 19/073,838, filed on Mar. 7, 2025.
Application No. 19/073,838 is a continuation of application No. PCT/US2024/016714, filed on Feb. 21, 2024.
(60) Provisional application No. 63/486,186, filed on Feb. 21, 2023.
(60) Provisional application No. 63/493,577, filed on Mar. 31, 2023.
(60) Provisional application No. 63/519,923, filed on Aug. 16, 2023.
(60) Provisional application No. 63/596,130, filed on Nov. 3, 2023.
(60) Provisional application No. 63/613,533, filed on Dec. 21, 2023.

(51) Int. Cl. A61M 3/02; A61K 9/08; A61K 31/155; A61K 31/19; A61K 31/194; A61K 31/549; A61K 31/7036; A61K 33/18; A61K 33/20; A61K 33/34; A61K 38/12; A61K 38/14

(52) U.S. Cl. CPC A61M 3/0233; A61K 9/08; A61K 31/155; A61K 31/19; A61K 31/194; A61K 31/549; A61K 31/7036; A61K 33/18; A61K 33/20; A61K 33/34; A61K 38/12; A61K 38/14

(57) ABSTRACT

Described herein are method and kits for locally treating or preventing infection in a patient in need thereof. The method may include locally administering to a human patient a plurality of doses of at least one antiseptic agent in solution by irrigating the at least one antiseptic agent at a wound, wherein the administration provides the patient continuous localized antiseptic therapy for at least a 24-hour period, at least 48-hour period, at least a 72-hour period, or at least a 96-hour period.

### CROSS-REFERENCE TO RELATED APPLICATIONS

This application is a continuation-in-part of U.S. application Ser. No. 19/073,838, filed Mar. 7, 2025, which is a continuation of International Application No. PCT/US2024/016714, filed Feb. 21, 2024, which claims priority to U.S. Provisional Application No. 63/486,186, filed Feb. 21, 2023, U.S. Provisional Application No. 63/493,577, filed Mar. 31, 2023, U.S. Provisional Application No. 63/519,923, filed Aug. 16, 2023, U.S. Provisional Application No. 63/596,130, filed Nov. 3, 2023, and U.S. Provisional Application No. 63/613,533, filed Dec. 21, 2023, the contents of each of which are incorporated herein by reference in their entirety.

### TECHNICAL FIELD

The present disclosure generally relates to methods, kits, doses, and concentrations of antifungal agent irrigation of biological tissue. In particular, the description relates to methods, kits, doses, and concentrations of irrigation of localized antifungal agents in and at wounds, including surgical wounds, traumatic wounds, wounds caused by or worsened by infection and wounds containing infected medical devices.

### BACKGROUND

Musculoskeletal wounds are a common, painful experience ranging in severity from minor abrasions and lacerations to major surgical or traumatic wounds encompassing severe injury to skin, subcutaneous tissues, and bone. The treatment of minor wounds may be partially addressed with topical treatments. Treatment of severe wounds, however, may involve surgical intervention, irrigation, repeated debridement, delayed healing, and subsequent closing of the wound. During extensive treatment of a severe wound, the wound is exposed to environmental contamination, including exposure of the wound to contamination from the skin or the environment over an extended period of time. Patients with extended wound exposure in the perioperative and postoperative period are more likely to develop an infection, which can be difficult to treat and may impair the patients' functional status and quality of life. Chronic infection is debilitating and may be life threatening.

The use of local antiseptics for the prevention and treatment of localized infections is common during open surgical procedures; however, local administration of antiseptic agents is very difficult after wound closure at the end of a surgical procedure and in chronic wounds that are covered with an occlusive dressing to prevent contamination of the wound.

In view of the foregoing, there is a substantial unmet clinical need for localized antiseptic therapy of surgical and chronic wounds (e.g., local antiseptic delivery at therapeutic concentrations for an extended period of time) after wound closure or coverage. There is also an unmet need for a new method for locally delivering an antiseptic agent for an extended period of time either alone or in combination with an additional antimicrobial agent.

### SUMMARY

In some aspects, provided herein is a method of treating or preventing a local infection in a human patient at a localized site, the method comprising locally administering by irrigation to the localized site of the human patient a plurality of doses of: at least one antiseptic agent in solution; and at least one antibiotic in solution.

In some aspects, the antiseptic agent comprises hypochlorous acid, sodium hypochlorite, citric acid, acetic acid, polyhexamethylene biguanide, chlorhexidine glutonate, povidone iodine, copper sulfate/potassium iodide, or taurolidine.

In some aspects, at least one antibiotic comprises vancomycin, daptomycin, and/or tobramycin.

In some aspects, the local infection comprises a bacterial pathogen, a fungal pathogen, or a polymicrobial pathogen.

In some aspects, irrigating the localized site comprises instillation of the at least one antiseptic agent in solution followed by a dwell period followed by aspiration of the at least one antiseptic agent in solution to remove the at least one antiseptic agent in solution. In some aspects, about 50 mL of the at least one antiseptic agent in solution is instilled with about a 5-minute dwell period followed by about a 5-minute aspiration.

In some aspects, the least one antiseptic agent in solution is locally administered by irrigation prior to the at least one antibiotic in solution.

Also provided herein, in some aspects, is a method of treating or preventing a local infection in a wound, the method comprising: locally administering to a human patient a plurality of doses of at least one antiseptic agent in solution by irrigating the wound, wherein the locally administering is performed at least once in a 24-hour period.

In some aspects, the antiseptic agent comprises hypochlorous acid, sodium hypochlorite, citric acid, acetic acid, polyhexamethylene biguanide, chlorhexidine glutonate, povidone iodine, copper sulfate/potassium iodide, or taurolidine.

In some aspects, the infection comprises a bacterial pathogen, a fungal pathogen or a polymicrobial pathogen.

In some aspects, irrigating the wound with the at least one antiseptic agent in solution is performed prior to a daily local antibiotic irrigation. In some aspects, irrigating the wound comprises instillation of the at least one antiseptic agent in solution followed by a dwell period followed by aspiration of the at least one antiseptic agent in solution to remove the at least one antiseptic agent in solution. In some aspects, about 50 mL of the at least one antiseptic agent in solution is instilled with about a 5-minute dwell period followed by about a 5-minute aspiration.

In some aspects, a sterile fluid is irrigated after the at least one antiseptic agent in solution to remove or otherwise dilute the at least one antiseptic agent. In some aspects, the sterile fluid comprises normal saline.

In some aspects, irrigating the wound with the at least one antiseptic agent in solution is accomplished by simultaneously instilling and aspirating the at least one antiseptic agent in solution. In some aspects, at least about 100 mL, at least about 250 mL, at least about 500 mL, at least about 1000 mL or at least about 2000 mL of the at least one antiseptic agent in solution is irrigated. In some aspects, the at least one antiseptic agent in solution is irrigated in less than about 15 minutes.

Also provided herein, in some aspects, is a kit for treating a localized infection in a human patient, the kit comprising: at least one antiseptic agent; a treatment delivery system and/or irrigation device configured to locally irrigate the localized infection with at least one dose of the at least one antiseptic agent; and instructions for administering the at least one antiseptic agent.

### DESCRIPTION OF FIGURES

These and other aspects will now be described in detail with reference to the following figures.

FIG. 1A shows an example of a treatment delivery system and kit.

FIG. 1B shows a detailed view of the treatment delivery system and kit of FIG. 1A.

### DETAILED DESCRIPTION

The disclosed subject matter relates to locally administered antifungal agents to improve treatment or prevention of fungal infections in infected wounds. The dosing and method of safely administering repeated or periodic doses and concentrations of antifungal agents is disclosed herein. The method of administering doses of antifungal agents at concentrations greater than previously practiced levels of systemic therapy, while maintaining systemic exposure of the patient to levels at less than or equal to safe systemic concentration limits is also disclosed. The methods described herein meet the needs described herein above. For example, the methods described herein provide new opportunities for a previously unachievable way of safe and effective local delivery of therapeutic agents such as antifungal agents by an irrigation route of administration to maximize localized antifungal efficacy and minimize the risks associated with exposure of the wound to concentrated antifungals and the environment during therapy. In addition, the methods described herein provide reduced side effects that are typically seen with topical antifungals, such as irritation, itching, or a systemic inflammatory response.

Successful localized antifungal therapy is dependent upon the specific antifungal agent used, the exposure of the wound to the antifungal agent, including the dose, concentration, and duration of exposure, which are prescribed based on the mechanism of action and pharmacodynamic target of the antifungal agent. Antifungal efficacy in the clinical setting is determined by the patient's response to the therapeutic agent and confirmation of effective eradication of the infection. Antifungal agents are commonly used topically for a wide range of indications, and local instillation of antifungal agents in solution has been reported to treat fungal infections of certain organs (e.g., lung, kidney and bladder). However, because instillation is unidirectional, (none of the instilled antifungal agent is aspirated or flushed from the site of infection), and the unidirectional instillation doses are absorbed entirely in the tissue and bloodstream, local and systemic toxicity considerations limit the local dose and concentration that may be safely administered by instillation. Complications of commonly used local antifungals may be life threatening. For example, complications of amphotericin B toxicity include anaphylaxis and renal failure. Complications of fluconazole toxicity include anaphylaxis, hepatotoxicity, clinical hepatitis, myalgia, seizures, acute kidney injury, thrombotic thrombocytopenia purpura and in rare cases death. Therefore, the methods of the present disclosure may allow for administration of antifungals to patients that would otherwise be contraindicated or excluded from treating with antifungals. For example, the patients may be on immunosuppressants or have cancer, renal insufficiency, or other conditions that would otherwise affect systemic absorption of the antifungal agent. As such, provided herein are methods of treating a fungal infection for a human patient in need thereof, wherein the patent has renal insufficiency, cancer, immunosuppression, or a combination thereof. Without being limited by theory, the immunosuppression in the human patient may be due to a condition (e.g., an auto-immune disorder of the human patient) or due to immunosuppressing medication administered to the human patient. Also provided herein are methods treating a fungal infection for a human patient in need thereof, with either no risk or substantially no risk of ototoxicity to the human patient. Also provided herein are methods treating a fungal infection for a human patient in need thereof, with either no risk or substantially no risk of nephrotoxicity to the human patient.

The present disclosure is a significant improvement and departure over previous methods, because it includes one or more safe and effective doses of at least one antifungal agent delivered via irrigation (e.g., instillation and subsequent aspiration) to a localized wound (e.g., traumatic wound, surgical wound, ulcer) of the human patient in need thereof. Severe complications from use of systemic antifungal agents may be substantially reduced or eliminated by the use of localized antifungal agents. Such safe and effective localized antifungal therapy by irrigation is accomplished by local irrigation, that is instilling sufficiently high concentrations of the antifungal agent and allowing sufficient time for the antifungal agent to soak the wound to be effective, then aspirating the antifungal agent from the wound with sufficient frequency to maintain safe systemic exposure to the antifungal agent (e.g., avoid local and systemic toxicity). This irrigation cycle can then be repeated within the time before the antifungal agent effect wears off. As such, in one embodiment, one or more doses of at least one antifungal agent at or above the minimum effective dose delivered to the localized wound may be bidirectional instead of unidirectional. The bidirectional, instillation and aspiration cycles of the irrigation therapy may be sequential or staggered.

In an embodiment, the local, cyclic administration provides the patient continuous localized antifungal therapy for a period of time. Said continuous therapy may include continuous localized exposure to a therapeutic concentration of the at least one antifungal agent over the period of time.

The period of time for continuous localized antifungal therapy may be at least a 24-hour period, at least a 48-hour period, at least 3 days, at least 4 days, at least 5 days, at least 6 days, at least 7 days, at least 8 days, at least 9 days, or at least 10 days. The localized administration of the antifungal agent may allow for the patient to complete therapy for the underlying surgical procedure faster, reduce the number of surgeries needed by the patient, or reduce the amount of time the patient is on antimicrobial therapy. For example, localized administration of the antifungal agent by irrigation may have a shorter therapy time (i.e. on the order of days) as compared to the therapy time when administered topical, IV, or oral agents (i.e. on the order of weeks). In some aspects, the localized administration of the antifungal agent reduces the treatment time by at least one week, at least two weeks, at least three weeks, or at least four weeks. In some aspects, the localized administration of the antifungal agent reduces the mortality rate of the human patient by at least 50%, at least 40%, at least 30%, at least 25%, at least 20%, at least 15%, at least 10%, at least 5%, at least 4%, at least 3%, at least 2%, or at least 1% over a given time frame (e.g., 1 week, 2 weeks, 3 weeks, 4 weeks, 6 weeks, 8 weeks, 1 month, 2 months, 3 months, 4 months, 5 months, 6 months, or 12 months). In some aspects, the localized administration of the antifungal agent reduces the risk of fungal reinfection of the human patient by at least 50%, at least 40%, at least 30%, at least 25%, at least 20%, at least 15%, at least 10%, at least 5%, at least 4%, at least 3%, at least 2%, or at least 1% (e.g., 1 week, 2 weeks, 3 weeks, 4 weeks, 6 weeks, 8 weeks, 1 month, 2 months, 3 months, 4 months, 5 months, 6 months, or 12 months).

In an embodiment, the localized administration may be at a faster rate than would be safe to administer systemically. For example, the instillation of the antifungal agent may be over a period of greater than 200 mg/hr, which is a limit for safe intravenous administration of fluconazole. The disclosed method of local irrigation may safely administer the at least 1 antifungal agent hourly at a rate of at least 200 mg/30 min, or at least 200 mg/10 min, or at least 200 mg/5 min, or at least 200 mg/min, thereby increasing the time period of exposure to the at least one antifungal agent at maximum concentration and increasing the pharmacodynamic target of MIC/AUC or MBEC/AUC.

In another embodiment, the present disclosure is an improvement over the prior methods by providing antifungal concentrations and doses administered locally that exceed recommended safe systemic concentrations and doses, but do not provide unsafe systemic blood plasma concentration levels to the human patient. The irrigating may include soaking the localized site of fungal infection with the at least one antifungal agent for a time interval between each alternating instillation and aspiration cycle.

In other examples, the soak/dwell time may be a fixed time of 5 minutes or more, 10 minutes or more, 20 minutes or more, 30 minutes or more, 60 minutes or more, 120 minutes or more, 180 minutes or more, or 240 minutes or more. In other examples, the soak time may be less than 180 minutes, less than 120 minutes, less than 60 minutes, less than 30 minutes, or less than 20 minutes. In other examples, the soak/dwell time interval between each instillation and aspiration cycle may be less than 5 minutes, 5 minutes to 60 minutes, about 10 minutes to about 180 minutes, about 30 minutes to about 120 minutes, or about 60 to about 90 minutes. The aspiration cycle may be less than 180 minutes, less than 120 minutes, less than 90 minutes, less than 60 minutes, or less than 30 minutes. As the concentration of the at least one antifungal agent is increased, the time between the instillation and aspiration phases of irrigation may be decreased to maintain safe serum concentrations.

The present disclosure further provides methods for maintaining an effective concentration of the at least one antifungal agent at or above a minimum effective dose by repeating an irrigation cycle 2 or more, 3 or more, 4 or more, 5 or more, 6 or more, 7 or more, 8 or more, 9 or more, 10 or more, 11 or more, or 12 or more times a day, even hourly or more frequently, at the site of the localized wound of the human patient. In other examples, the irrigating is performed at least twice, at least 4 times, at least 8 times, at least 16 times, or continuously over a 24-hour period. Continuous irrigation includes sequential instillation and aspiration cycles without any time between a previous aspiration cycle and the next instillation.

The effectiveness of unidirectional instillation methods is limited by the potential toxicity of sustained concentrations of the antifungal agent delivered to attain the pharmacodynamic target. Local irrigation, adopted to specific pharmacodynamics and pharmacokinetics of each antifungal agent overcomes this prior limitation. Several classes of antifungal agents may be appropriate for local irrigation of wounds, including but not limited to: azole derivatives from the group consisting of fluconazole, isavuconazole, posaconazole, voriconazole, and combinations thereof, amphotericin B or lipid formulations thereof, and echinocandins selected from the group consisting of anidulafungin, caspofungin, and micafungin. It can be seen by those skilled in the art that each of the aforementioned antifungal agents may be administered using the irrigation methods provided herein to provide safe and effective antifungal therapy of a wound by adjusting the volume, concentration, soak time, time to evacuation and cycle frequency best suited to the pharmacodynamics and pharmacokinetics of the antifungal agent to be administered.

The present methods allow for administering a greater total daily dose of at least one antifungal agent than what is recommended for safe systemic administration. In an example, the locally administered dose of the antifungal agent in a 24-hour period exceeds the recommended daily systemic dose of the antifungal agent for an adult human patient for a 24-hour period. The recommended daily dose may be a dose recommended for administration to the adult human patient intravenously, orally or by injection. The daily dose or total amount may be the sum of the plurality of doses administered in the period of time. In some embodiments, the daily dose/amount provided by local administration may be ≥10 mg/day, ≥50 mg/day, ≥100 mg/day, ≥200 mg/day, ≥300 mg/day, ≥400 mg/day, ≥500 mg/day, ≥600 mg/day, ≥700 mg/day, ≥800 mg/day, ≥900 mg/day, ≥1000 mg/day, ≥1500 mg/day, ≥2000 mg/day, ≥2500 mg/day, ≥3000 mg/day, ≥4000 mg/day or ≥5000 mg/day of at least one antifungal agent. In some examples, the antifungal agent may be locally administered for at least a 12-hour period and the daily dose/amount provided by the local administration may be about half the amount that would be locally administered in a 24-hour period.

In other embodiments, the present methods allow for administering a human patient a plurality of doses of at least one antifungal agent in solution by irrigating the at least one antifungal agent at a wound. The administration provides the patient continuous localized antifungal therapy for at least a 4-hour period, a 8-hour period, a 12-hour period, a 16-hour period, a 20-hour period, or a 24-hour period. In some examples, the administration provides a total daily dose less than or equal to established FDA approved intravenous doses of the antifungal agent.

In some examples, the locally administered dose (total amount) of fluconazole, isavuconazole, posaconazole, or voriconazole is at least 100 mg, at least 150 mg, at least 200 mg, at least 250 mg, at least 300 mg, at least 400 mg, at least 500 mg, at least 550 mg, at least 600 mg, at least 700 mg, at least 800 mg, at least 900 mg, at least 1000 mg, at least 1500 mg, at least 2000 mg, at least 2500 mg, or at least 3000 mg in a 24-hour period. In other examples, the locally administered daily dose of fluconazole is 100 to 3,000 mg, at least 200 mg to 1,500 mg, or at least 1,000 mg to 3,000 mg in a 24-hour period. In other embodiments, the maximum daily dose provided by local administration of anidulafungin, caspofungin, or micafungin may be >50 mg/day, >100 mg/day, >200 mg/day, >300 mg/day, >400 mg/day, >500 mg/day, >750 mg/day, or >1,000 mg/day. In an example, the locally administered dose of micafungin is at least 100 mg or at least 800 mg, at least 200 mg, at least 400 mg, in a 24-hour period. In an example, the locally administered dose/amount of amphotericin B is at least 15 mg, at least 25 mg, at least 50 mg, at least 100 mg, at least 250 mg, at least 500 mg, or at least 750 mg in a 24-hour period.

Table 1 provides example local antifungal dosage and concentration ranges. Table 1 provides example local concentrations for antifungal irrigation therapy in 1200 mL solution (if administered 50 ml/hour). In additional examples, the volume administered may be less than or more than 50 cc, and the resulting concentration would be proportionally greater than or less than the concentrations listed in Table 1.

TABLE 1

| | Local Daily | |
|---|---|---|
| Drug | Dosage Range | Local Concentration Range |
| Fluconazole | 100 mg-2400 mg | 83 mcg/ml-2,000 mcg/ml |
| Isavuconazole | 136 mg-2000 mg | 113 mcg/ml-74400 mcg/ml |
| Voriconazole | 100 mg-2000 mg | 83 mcg/ml-10,000 mcg/ml |
| Amphotericin B | 25 mg-1000 mg | 20 mcg/ml-10,000 mcg/ml |
| Anidulafungin | 50 mg-1000 mg | 41 mcg/ml-3300 mcg/ml |
| Caspofungin | 50 mg-1000 mg | 41 mcg/ml-5000 mcg/ml |
| Micafungin | 50 mg-1000 mg | 41 mcg/ml-10,000 mcg/ml |

The volume of locally administered antifungal agent may be sufficient to allow the entire surface of the wound to be exposed throughout the soak period. The volume of the administered dose for each soaking step may be about 5 cc, about 10 cc, about 20 cc, about 30 cc, about 40 cc, about 50 cc, about 60 cc, about 70 cc, about 80 cc, about 90 cc, about 100 cc, or greater than 100 cc. In some aspects, the continuous antifungal treatment may be provided within a subcutaneous wound volume of at least 5 cc, at least 10 cc, 25 cc, 50 cc, 75 cc or 100 cc, and may be provided at a depth beneath the skin of at least 1 cm, 2 cm, 3 cm, 5 cm, 10 cm, or 15 cm (e.g., a small or large anatomical joint space or a small or large surgical wound). In other examples, the continuous antifungal treatment may be delivered topically/superficially to local wound beds up to superficial layers.

The irrigating may be performed at least once daily, at least twice daily, at least 5 times daily, at least 10 times daily, at least 15 times daily, at least 20 times daily, at least 21 times daily, or at least 22 times daily. In one example, an antifungal agent may be irrigated at the wound site every hour for 20 to 22 hours. In some examples, two or more antifungal agents may be irrigated at the wound site concurrently or sequentially. The two or more antifungal agents may be irrigated at different frequencies. For example, one may be irrigated hourly for 12 to 24 hours and a second may be irrigated 1 to 2 times per day.

The daily dose of the at least one antifungal agent may substantially exceed the maximum recommended daily systemic dose of the at least one antifungal agent. Substantially exceeding the maximum recommended daily systemic dose of the at least one antifungal agent may include the daily dose exceeding the maximum recommended daily systemic dose by at least 110%, at least 125%, at least 150%, at least 175%, at least 200%, at least 225%, at least 250%, at least 275%, at least 300%, at least 400%, at least 500%, at least 600%, at least 700%, at least 800%, at least 900%, at least 1,000% or greater.

In some embodiments, the at least one antifungal agent is fluconazole. The locally administered concentration of fluconazole may be at least 0.2 mg/mL, at least 0.4 mg/mL, at least 0.8 mg/mL, or at least 1.6 mg/mL. The maximum recommended daily systemic dose of fluconazole is 400 mg/day. The daily dose of locally irrigated fluconazole may be 100 mg to 200 mg, 200 mg to 400 mg, 400 mg to 800 mg, 800 mg to 1,200 mg, 1,200 mg to 1,600 mg, or more than 1,600 mg. The total dose of fluconazole administered over all administration days may range from about 1 g to 2 g, about 2 g to 4 g, about 4 g to 8 g, about 8 g to 12 g, about 12 g to 16 g, or more than 16 g.

Although a high concentration is administered locally, the benefit of the disclosed methods is that the serum concentration for the antifungal agent is maintained at a level below the maximum safe level. Therefore, the patient receives the benefit of high concentrations of the antifungal agent at the site of infection while maintaining safe serum levels and minimizing side effects to the patient. Serum concentration is the concentration of the antifungal agent in the blood serum of the patient from an intravenous sample from anywhere in the body and is used to monitor systemic toxicity. The maximum safe serum concentration of the antifungal agent may be about 50 mcg/mL. In some examples, the serum concentration of the antifungal agent when administered according to the disclosed methods is less than 10 mcg/mL, less than 5 mcg/mL, less than 4 mcg/mL, less than 3 mcg/mL, less than 2.5 mcg/mL, less than 2 mcg/mL, or less than 1 mcg/mL. In an example, the maximum safe serum concentration of voriconazole may be about 50 mcg/mL. In some examples, the serum concentration of fluconazole when administered according to the disclosed methods is less than 5 mcg/mL, less than 4 mcg/mL, less than 3 mcg/mL, less than 2.5 mcg/mL, less than 2 mcg/mL, or less than 1 mcg/mL.

In some aspects, the method may optionally include monitoring the serum concentration of the antifungal agent. In at least one embodiment, the serum level or serum concentration of the antifungal agent is undetectable. In some embodiments, the administering provides a serum concentration of the antifungal agent of less than 5 mcg/mL, less than 2.5 mcg/mL, less than 1.25 mcg/mL, or less than the limit of detection or quantification for the test method used in the human patient. In an example, the locally administered dose of voriconazole is at a concentration of at least 100 mcg/mL and the administering provides a serum concentration of voriconazole of less than 0.5 mcg/mL in the human patient and is below the limit of detection or limit of quantification. In another example, the locally administered dose of voriconazole is at a concentration of at least 800 mcg/mL and the administering provides a serum concentration of voriconazole of less than 5 mcg/mL in the human patient. In yet another example, the locally administered daily dose of voriconazole is 100 to 3,000 mg in a 24-hour period, and the administering provides a serum concentration of fluconazole of less than 5 mcg/mL in the human patient.

The methods described herein may locally treat fungal infection at a wound in a patient in need thereof. The radius of the wound may be at least 1 cm, at least 5 cm, at least 10 cm, or at least 15 cm from the center of the wound (or the site of localized administration). In some examples, the irrigation may occur within a two-dimensional or three-dimensional perimeter of the wound. In some examples, the wound may be a topical wound or a subcutaneous wound. For example, the fungal infection may include a topical species of fungus, a subcutaneous species of fungus, or a combination thereof, most commonly including candida albicans, candida parapsilosis and aspergillus, and more rarely including dimorphic fungi, pigmented yeast, dematiaceous fungi and other filamentous fungi. In other examples, the wound may be a chronic wound. Chronic wounds include a wide variety of wounds that may be grouped into categories, including traumatic wounds, surgical wounds, ulcers of various causes and burns. Traumatic wounds include abrasions and lacerations, penetration wounds, compound fractures and blast injuries. Chronic surgical wounds can occur at any operative site and include incisional wounds and deep surgical wounds, including fistulas. Chronic ulcers include pressure sores, diabetic foot ulcers, vasculitic ulcers, arterial ulcers, and venous stasis ulcers. Chronic wounds from burns may occur on any part of the body and vary greatly in size and severity. Surgical wounds and traumatic wounds previously treated surgically may also include implanted medical devices within the wound. For example, the surgical wound may be a subcutaneous surgical wound and the subcutaneous surgical wound may include an implanted medical device in some instances. In some examples, the implanted medical device may be an orthopedic implant, such as a joint replacement prosthesis.

In some embodiments, the method may treat or prevent persistent infection, reinfection, or mortality of the patient. In an example, the method may prevent the occurrence of a persistent infection by completely irradicating the fungal infection after the administering is complete. In another example, the method may prevent reinfection by locally administering the antifungal agent after a prior antifungal infection has been treated to prevent further infection. In yet another example, the method may prevent mortality or reduce the likelihood of mortality by treating or preventing a fungal infection in a wound.

In some embodiments, the method may further include locally administering at least one additional therapeutic agent for treatment of a condition other than fungal infection. Chronic wounds are frequently complicated by the presence of polymicrobial infection. Musculoskeletal infections are often polymicrobial and biofilm forming, and identification of the pathogen(s) typically occurs using tissue cultures 7-14 days after initiating therapy. In a significant minority of cases, the pathogens are never identified by tissue culture methods, and recent research employing genetic sequencing techniques have identified multiple organisms both in culture negative cases and cases with a single pathogen identified by tissue culture. In some examples, the at least one additional therapeutic agent is at least one antimicrobial agent. So, administration of one or more antimicrobial agents in addition to an antifungal agent would provide additional benefit in localized wound management.

Broad spectrum systemic antibiotic therapy is the standard first-line treatment of localized infections, particularly when the pathogen is not yet identified or may be polymicrobial. In the case of infections in the presence of an implanted device such as periprosthetic joint infection (PJI), breast implant infection (BII), or fracture related infection (FRI), broad spectrum therapy exceeding the biofilm bactericidal concentration (BBC) or minimum biofilm eradication concentration (MBEC) for all identified or potential pathogens is preferred. Pre-clinical research to quantify BBC and MBEC of antibiotics for the pathogens found in local infections of humans has not been comprehensive. The number of potential antibiotics and known pathogens is large, and the BBC and MBEC values identified to date generally far exceed safe systemic dosing and concentrations of the antimicrobial agents tested, making the practical clinical application of such research unlikely. In many reports where testing to establish MBEC values was limited to clinically recommended dosing or concentrations, no concentration of antimicrobial tested was found to eradicate a biofilm of a specific organism, such that the MBEC for the specific antibiotic and strain of organism tested remains unknown. Local irrigation of antimicrobials at concentrations above MBEC for common wound infections while maintaining safe serum concentrations is therefore desirable.

Broad spectrum antibiotic therapy for localized infections commonly includes a glycopeptide (e.g., vancomycin HCl) for coverage of gram+ bacteria and an aminoglycoside (e.g., gentamicin or tobramycin sulfate) for coverage of gram− bacteria. Examples of safe dosing and systemic concentrations of these and other antibiotics are shown in Table 2.

TABLE 2
Safe Systemic Dose and Concentration of Select Antibiotics

| | |
|---|---|
| Safe Daily Dose | Safe IV Concentration |

| | | | | |
|---|---|---|---|---|
| Amikacin | 15 | mg/kg/day | 5 | mg/mL |
| Gentamicin | 7 | mg/kg/day | 0.2 | mg/mL |
| Tobramycin | 7 | mg/kg/day | 5 | mg/mL |
| Cefazolin | 6 | g/day | 330 | mg/mL |
| Cefepime | 6 | g/day | 40 | mg/mL |
| Ampicillin | 12 | g/day | 125 | mg/mL |
| Ciprofloxacin | 1.2 | g/day | 2 | mg/mL |
| Azithromycin | 0.5 | g/day | 2 | mg/mL |
| Doxycycline | 0.2 | mg/day | 1 | mg/mL |
| Clindamycin | 2.7 | g/day | 18 | mg/mL |
| Daptomycin | 10-12 | mg/kg/day | 8-50 | mg/mL |
| Vancomycin | 45-60 | mg/kg/day | 5 | mg/mL |

In polymicrobial infections, the pathogens involved may include Gram+ and Gram − bacteria, fungi, or any combinations of these pathogens. In such cases, as well as in cases of culture negative infections (i.e., no pathogen identified), it is best practice to administer more than one antimicrobial agent for the broadest possible coverage. What is needed clinically in addition to adequate and safe antibiotic therapy, is a systemically safe antifungal therapy which provides localized biofilm eradication under highly variable clinical conditions.

Antifungal therapy for localized infections may include: an azole derivative from the group consisting of fluconazole, isavuconazole, posaconazole, voriconazole, and combinations thereof; amphotericin B or lipid formulations thereof; or an echinocandin selected from the group consisting of anidulafungin, caspofungin, and micafungin. Examples of safe dosing and systemic concentrations of these antifungal agents are shown in Table 3.

TABLE 3
Safe Systemic Dose and Concentration of Select Antibiotics

| Drug | Safe Daily Dose | | Safe IV Concentration | |
|---|---|---|---|---|
| Fluconazole | 800 | mg | 2,000 | mcg/ml |
| Isavuconazole | 1116 | mg | 1488 | mcg/ml |
| Voriconazole | 600 | mg | 5000 | mcg/ml |
| Amphotericin B | 1.5 mg/kg (150 mg) | | 250 | mcg/ml |
| Anidulafungin | 200 | mg | 1000 | mcg/ml |
| Caspofungin | 70 | mg | 500 | mcg/ml |
| Micafungin | 150 | mg | 1500 | mcg/ml |

At least one antibacterial agent may be administered locally by irrigation, in a manner similar to the irrigation of the at least one antifungal agent. The at least one antifungal agent may be administered sequentially and/or concurrently with the at least one antibacterial agent. In some embodiments, the at least one antibacterial agent is at least one antibiotic. Various antibiotics used to treat infections and biofilms may be used with the disclosed methods. In some embodiments, the at least one antibacterial agent is two or more antibiotics. The two or more antibiotics may be sequentially administered. In some examples, a first antibiotic may be simultaneously (concurrently) administered with the at least one antifungal agent and a second antibiotic may be sequentially administered after the administration of the first antibiotic and the at least one antifungal agent.

There may be minimal or no drug-drug interactions between the at least one antifungal agent and the at least one antibiotic administered. Because the agents are administered locally by irrigation, they are removed locally before there is significant systemic absorption or metabolism. Therefore, there is less opportunity for either agent to interact systemically with each other or any other medications the patient may be taking. The disclosed method may provide for combinations of antifungals, antibiotics, anesthetics, analgesics, or other drugs to be administered that could not otherwise be combined systemically. For example, the combined local administration of the at least one antifungal agent and the at least one antibacterial agent may not be nephrotoxic or may not be ototoxic to the patient.

The at least one antibiotic may be selected from vancomycin, daptomycin, tobramycin, gentamicin, or other antibiotics known in the art. In some embodiments, the two or more antibiotics may include vancomycin and tobramycin and the at least one antifungal agent may be fluconazole. In an example, the fluconazole and vancomycin may be administered concurrently, and the tobramycin may be administered sequentially to the concurrently administered fluconazole and vancomycin, while the serum concentrations of each of the fluconazole, the vancomycin and the tobramycin remain below the recommended safe serum concentrations of each of the fluconazole, the vancomycin and the tobramycin throughout the time-period of administration.

As a route of administration, irrigation is defined as administration to bathe or flush open wounds or body cavities. By controlling irrigation dose, that is, concentration, volume, and duration of exposure (bathing or flushing) time, a novel method of treating or preventing localized fungal infections in a human patient has been developed. The irrigation concentration or the total irrigation dose of the antifungal agent may exceed manufacturer's recommended concentration. Moreover, the dose and concentration to achieve BBC or MBEC for biofilm forming organisms varies depending upon the organism, the maturity of the biofilm, the location of the biofilm, the implant or tissue to which the biofilm has attached, the dilution and absorption rate of the antifungal agent at the site of irrigation and other factors. In one aspect, provided herein are methods of treating or preventing a localized fungal infection in a wound of a human patient in need thereof. The novel method described herein includes sustained administration of one or more antimicrobial agents, preferably at least one antifungal agent, by an irrigation route of administration. Optimal efficacy of this novel method is obtained when the dose and concentration of each antimicrobial agent is administered based on the pharmacodynamics and pharmacokinetics of the drug to be administered. In one aspect of the invention, one or more of the drugs to be administered by irrigation is administered at a dose and/or concentration exceeding previously established safe systemic dose and/or concentrations of said drug(s), while providing safe systemic (serum) concentrations of said drug(s). Non-limiting examples of antifungal agents include fluconazole, isavuconazole, posaconazole, voriconazole, amphotericin B or lipid formulations thereof, anidulafungin, caspofungin, micafungin, and combinations thereof.

The daily dose of the at least one antifungal agent may comprise a single administration or a plurality of administrations during the day. For example, the daily dose may be administered as 1, 2, 3, 4, 5, 6, 7, 8, 9, 10, 11, 12, 13, 14, 15, 16, 17, 18, 19, 20, 21, 22, 23, or 24 administrations during the day. In some examples, the daily dose of at least one antifungal agent and at least one additional antimicrobial agent may be administered sequentially and/or concurrently over at least 1, at least 2, at least 5, at least 10, at least 15, at least 20, or at least 24 administrations. The administrations may be every 30 minutes or less, about every hour, about every 2 hours, about every 3 hours, about every 4 hours, about every 5 hours, about every 6 hours, about every 7 hours, about every 8 hours, about every 9 hours, about every 10 hours, about every 12 hours, about every 18 hours, or about every 24 hours.

The daily dose may also comprise a plurality of administrations such that at least one antifungal agent is administered in series with at least one antibiotic, and at least one dose of combined therapy for fungal and bacterial infection of the wound is considered the totality of both serial administrations. Alternatively, the daily dose may comprise a once daily administration of at least one antimicrobial agent, and the daily dose of combined therapy may include a single administration or a plurality of administrations during the day. In an embodiment, at least one antifungal agent may be administered concurrently with at least one additional antimicrobial agent, followed by administration of at least one additional antimicrobial agent in series to achieve both antifungal and broad-spectrum antibacterial coverage. Specifically, one embodiment may include concurrent administration of fluconazole and vancomycin (or daptomycin) in series with administration of tobramycin (or gentamicin). This particular irrigation regimen is tailored to the pharmacodynamic targets of each administered drug, specifically tobramycin C_{Max}/MIC, vancomycin AUC//MIC and fluconazole AUC/MIC. In another embodiment may include sequential administration of amphotericin B with at least one antimicrobial agent (e.g. tobramycin).

Alternatively, the daily dose may include a once daily administration of at least one antifungal agent, followed in series by a plurality of administrations of at least one additional therapeutic agent for the remainder of the day. In an embodiment, the once daily administration includes concurrently administering fluconazole and tobramycin, which is allowed to soak 2 hours followed by 30 minutes aspiration and the plurality of serial administrations includes sequential administration of vancomycin irrigations, which are allowed to soak for 30 minutes followed by 30 minutes aspiration and repeated for the remainder of the day, at which time, the once daily concurrent administration of fluconazole and tobramycin is repeated to start a new day of combined therapy.

Irrigating the wound may include soaking the biological tissue of the wound with the antifungal agent for about 10 minutes to about 180 minutes, about 30 minutes to about 180 minutes, about 30 minutes to about 45 minutes, about 30 minutes to about 60 minutes, about 60 minutes to about 90 minutes, about 60 minutes to about 120 minutes, or about 120 minutes to about 180 minutes. In various examples, irrigating the wound may include soaking with the antibiotic for 30 minutes, 60 minutes, 90 minutes, 120 minutes, 150 minutes, or 180 minutes. In the case of fluconazole, a 30-minute soak time may be optimal. In other cases, the optimal soak time will depend upon administered concentration and may be adjusted to administer higher concentrations for effective fungal eradication, while maintaining safe levels of systemic exposure as determined by serum concentrations or other serum levels, such as creatinine.

Administration of localized antifungal agents, with or without additional antimicrobial agents may be performed for a single day or a plurality of days as needed to provide the needed treatment or prevention. Irrigation may be performed for 1 or more days, 2 or more days, 3 or more days, 4 or more days, 5 or more days, 6 or more days, 7 or more days, 8 or more days, 9 or more days, 10 or more days, 14 or more days, or 30 or more days. The local administration may be repeated for additional periods of up to 10 days (e.g., with a change of the irrigation apparatus between repeated administration periods).

Further provided herein is a method of treating or preventing localized fungal infection in a wound of a human patient in need thereof by locally administering by irrigation to the human patient repeated doses of at least two therapeutic agents from separate sources without interruption to change sources. In an embodiment, one of the two or more therapeutic agents is at least one antifungal agent. In another embodiment, one of the two or more therapeutic agents is at least one antifungal agent and at least one additional antimicrobial agent. In some examples, the two or more therapeutic agents are administered sequentially, concurrently, or combinations thereof. The at least one additional antimicrobial agent may be at least one antibiotic or at least two antibiotics. For example, the at least one antifungal agent is fluconazole and the at least two antibiotics are vancomycin and tobramycin. The fluconazole and vancomycin may be administered concurrently from a first source, and the tobramycin may be administered from a second source sequentially with the concurrent fluconazole and vancomycin. The fluconazole and vancomycin are administered from a common source. In a further embodiment, the sequentially administered at least two therapeutic agents may be administered without human intervention to change between the first source and the second source.

Further provided herein is a method of treating or preventing fungal infection of a localized wound of a human patient by locally administering by irrigation to the human patient a concentration of at least one antifungal agent that exceeds the recommended concentration or maximum recommended daily dose of the at least one antifungal agent such that the local administration by irrigation provides a serum concentration that is less than the maximum safe serum concentration of the at least one antifungal agent.

In an embodiment, the method may include locally administering to the human patient a dose of at least one antifungal agent that substantially exceeds the recommended safe dose of the antifungal agent, and providing serum concentrations less than or equal to a maximum safe serum concentration of the antifungal agent. In some aspects, a recommended safe dose is the maximum recommended systemic daily dose.

In one method, 50 cc of fluconazole may be administered hourly for 22 hours for 7 days at a concentration of 400 mcg/mL, concurrently with vancomycin at a concentration of 2,500 mcg/mL and sequentially with 50 cc of tobramycin administered once daily at a concentration of 1,600 mcg/mL. The daily doses in this method are 440 mg fluconazole, 80 mg tobramycin and 3,000 mg of vancomycin, and the total doses are about 3.08 g of fluconazole, about 0.56 g of tobramycin and about 21 g of vancomycin, fluconazole fixed dose ratios of 5.5:1 and 0.18:1 respectively with tobramycin and vancomycin. The maximum doses and concentrations that may be administered are calculated pharmacokinetically based on the selected daily exposure and known values of pharmacokinetic parameters, and such doses and concentrations may be adjusted empirically prior to or during administration in the case of a patient with abnormal absorption or elimination of the antibiotic.

In one method, one or more antifungal agents may be administered at a previously known safe dose in concentration(s) that exceed previously known safe concentrations of such antifungal agents while providing clinically established safe systemic levels of said antifungal agents. This is accomplished by irrigation of the wound at higher concentrations less frequently per day, so as to administer a daily dose that is maintained within previously known safe limits. In an alternate method, one or more antifungal agents may be administered in a daily dose that exceeds previously known safe daily doses in concentration(s) within known safe limits while providing clinically established safe systemic levels of the one or more antifungal agent. This is accomplished by irrigation of the wound within previously known safe concentration limits more frequently per day so as to administer a daily dose that exceeds previously known safe limits. In either case, safe serum levels may be maintained by adjusting the soak time and evacuation time for each dosing cycle to limit the systemic absorption of each administration of one or more antifungal agent.

When two or more therapeutic agents are administered in the methods described herein, they may be administered at a fixed dose ratio less than, greater than or equal to 1:1; however, it may be beneficial to administer therapeutic agents at fixed dose ratios exceeding the maximum dose ratios previously established. For example, based on the 5^{th} and 95^{th} percentile of patient weight in the U.S., the minimum and maximum recommended daily systemic dose of vancomycin is about 1,200 g and 7,500 g respectively, and the minimum and maximum recommended daily systemic dose of tobramycin is about 200 g and 1250 g respectively. The resulting recommended fixed dose ratio of vancomycin to tobramycin is therefore 6:1. The methods disclosed herein describe administration of antibiotics at or exceeding MBEC for the target organisms while maintaining safe systemic levels of the antibiotics. In such cases, the fixed dose ratios may exceed previously known systemic dose ratios. In one method, the dose ratio of vancomycin to tobramycin is 40:1. In an alternate method, the fixed dose ratio of vancomycin to tobramycin is 125:1. In another alternate method, the fixed dose ratio of vancomycin to tobramycin is 10:1. In one embodiment, similar ranges of fixed dose ratios are provided for two antifungal agents. In another embodiment, similar ranges of fixed dose ratios are provided for at least one antifungal agent and at least one antibiotic.

Not all therapeutic agents may be administered simultaneously, due to incompatibility of the specific therapeutic agents or differences in their pharmacodynamic targets. For example, tobramycin is administered for the optimal concentration dependent target with an optimal exposure of 2 hours at therapeutic concentration, whereas vancomycin is administered with an optimal pharmacodynamic target of AUC/MIC or total daily exposure. It is a further benefit of the methods and irrigation route of administration described herein to enable the concurrent or serial administration of multiple solutions at or above therapeutic targets from multiple sources without interruption or input from the patient or user to change sources of the otherwise incompatible therapeutic agents.

Doses and concentrations and methods described herein may be described in the context of a particular wound, yet other wounds or conditions are considered as well. For example, where a method is described as useful in periprosthetic joint infection, the method may be used in breast implant infection, another infected wound such as fracture related infection, a diabetic ulcer, osteomyelitis, surgical site infection, etc. The antifungal agents and methods described herein may be configured for use in any of a variety of localized wounds and treatment sites beyond those specifically called out herein.

The treatment methods described herein may be performed manually (e.g., instillation by syringe and removal of fluids via a gravity drain), semi-automatically (e.g., instillation by syringe pump and aspiration by an active drainage device) or automatically (e.g., via a purpose-built irrigation delivery system). The irrigation delivery system may control irrigation (e.g., localized antifungal delivery and removal) according to a set protocol including one or multiple sequential phases for delivery at one or multiple times and durations per day.

Also provided herein, in some aspects, is a method of treating or preventing a local infection in a wound, the method comprising: locally administering to a human patient a plurality of doses of at least one antiseptic agent in solution by irrigating the wound, wherein the locally administering is performed at least once in a 24-hour period. In some aspects, the infection may include a bacterial pathogen, a fungal pathogen or a polymicrobial pathogen.

In some aspects, the antiseptic agent comprises hypochlorous acid, sodium hypochlorite, citric acid, acetic acid, polyhexamethylene biguanide, chlorhexidine glutonate, povidone iodine, copper sulfate/potassium iodide, or taurolidine. In some aspects, the antiseptic agent is a class or subset of antifungal agents that exhibit broad-spectrum activity against bacteria, viruses, or other microbes in addition to fungi.

In some aspects, irrigating the wound with the at least one antiseptic agent in solution is performed prior to a local antimicrobial irrigation. In some aspects, irrigating the wound with the at least one antiseptic agent in solution is performed following a daily local antimicrobial irrigation. In other aspects, the at least one antiseptic agent in solution is locally administered by irrigation concurrently with the local antimicrobial irrigation. The daily local antimicrobial irrigation may include irrigation of at least one antibiotic. The at least one antibiotic may include but is not limited to vancomycin, tobramycin, or any other antibiotic disclosed herein.

In some aspects, the wound is irrigated with about 10 mL to about 2000 mL of the antiseptic solution, such as about 10 mL to about 1000 mL, about 20 mL to about 500 mL, about 30 mL to about 250 mL, about 40 mL to about 100 mL, about 100 mL to about 300 mL, about 200 mL to about 400 mL, about 300 mL to about 500 ml, about 500 mL to about 1,000 mL, about 1,000 mL to about 1,500 mL, or about 1,500 mL to about 2,000 mL of the antiseptic solution. In some aspects, the wound is irrigated with about 10 mL, about 20 mL, about 30 mL, about 50 mL, about 60 mL, about 70 mL, about 80 mL, about 90 mL, about 100 mL, about 200 mL, about 300 mL, about 400 mL, about 500 mL, about 600 mL, about 700 mL, about 800 mL, about 900 mL, about 1,000 mL, about 1,100 mL, about 1,200 mL, about 1,300 mL, about 1,400 mL, about 1,500 mL, about 1,600 mL, about 1,700 mL, about 1,800 mL, about 1,900 mL, or about 2,000 mL of the antiseptic solution. In some aspects, irrigating the wound comprises instillation of about 50 mL of the antiseptic solution.

In some aspects, irrigating the wound comprises instillation of the at least one antiseptic agent in solution followed by a dwell period followed by aspiration of the at least one antiseptic agent in solution to remove the at least one antiseptic agent from the wound. In some aspects, the dwell period is about 1 minute to about 30 minutes, such as about 1 minute to about 25 minutes, about 1 minute to about 20 minutes, about 1 minute to about 15 minutes, about 1 minute to about 10 minutes, or about 1 minute to about 5 minutes. In some aspects, the dwell period is about a 5-minute dwell period. In some aspects, the aspiration is about 1 minute to about 30 minutes, for example, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, or about 30 minutes. In some aspects, the at least one antiseptic agent in solution is instilled with about a 5-minute dwell period followed by about a 5-minute aspiration. In some aspects, about 50 mL of the at least one antiseptic agent in solution is instilled with about a 5-minute dwell period followed by about a 5-minute aspiration.

In some aspects, a sterile fluid is irrigated after the at least one antiseptic agent in solution to remove or otherwise dilute the at least one antiseptic agent. In some aspects, the sterile fluid comprises normal saline.

In other aspects, irrigating the wound with the at least one antiseptic agent in solution is accomplished by simultaneously instilling and aspirating the at least one antiseptic agent in solution. In some aspects, at least about 100 mL, at least about 250 mL, at least about 500 mL, at least about 1000 mL, or at least about 2000 mL of the at least one antiseptic agent in solution is irrigated by simultaneous instillation and aspiration.

In some aspects, the at least one antiseptic agent in solution is irrigated in less than about 30 minutes, less than about 25 minutes, less than about 20 minutes, less than about 15 minutes, less than about 10 minutes, or less than about 5 minutes. In some aspects, the at least one antiseptic agent in solution is irrigated in less than about 15 minutes.

Also provided herein, in some aspects, is a method of treating or preventing a local infection in a human patient at a localized site, the method comprising locally administering by irrigation to the localized site of the human patient a plurality of doses of: i) at least one antiseptic agent in solution; and ii) at least one antibiotic in solution. In some aspects, the antiseptic agent comprises hypochlorous acid, sodium hypochlorite, citric acid, acetic acid, polyhexamethylene biguanide, chlorhexidine glutonate, povidone iodine, copper sulfate/potassium iodide, or taurolidine. In some aspects, the antibiotic is selected from the group consisting of amikacin, gentamicin, tobramycin, cefazolin, cefepime, ampicillin, ciprofloxacin, azithromycin, doxycycline, clindamycin, daptomycin, vancomycin and combinations thereof. In some aspects, the at least one antibiotic comprises tobramycin sulfate and/or vancomycin hydrochloride. In some aspects, the infection comprises a bacterial pathogen, a fungal pathogen or a polymicrobial pathogen. In some aspects, the at least one antiseptic agent in solution is locally administered by irrigation prior to the at least one antibiotic in solution. In other aspects, the at least one antibiotic in solution is locally administered by irrigation prior to the at least one antiseptic agent in solution. In other aspects, the at least one antibiotic in solution is locally administered by irrigation in combination with the at least one antiseptic agent in solution.

Also provided herein is an irrigation apparatus for treating or preventing a localized wound of a human patient in need thereof. The irrigation apparatus may be configured to control the irrigating automatically. The irrigation apparatus may be configured to locally administer a daily dose of at least one antifungal agent for up to 24 hours without interruption or input from the patient or user. In an embodiment, the irrigation apparatus may be configured to include 2 or more independent sources of therapeutic agents from which the irrigation apparatus draws the therapeutic agents according to various cycle times and frequencies described herein. In some embodiments, the irrigation apparatus delivers the therapeutic agents from the independent sources sequentially, concurrently, alternatingly, or periodically without any human intervention to change sources, connect the irrigation apparatus to each source or otherwise access or control flow of the therapeutic agents according to the various cycle times and frequencies. In an example, a first independent source (e.g., bag of solution) may include at least one antifungal agent and a second independent source (e.g., bag of solution) may include at least one antibiotic. In another example, a first independent source may include at least one antifungal agent and at least one antibiotic and a second independent source may include at least one antibiotic or at least one other therapeutic agent.

In some embodiments, the irrigation apparatus may further include an aspirating component. The aspirating component may be configured to apply suction to or around the wound to remove any previously administered therapeutic agent from the infection site. For example, the aspirating component may be configured to apply vacuum of at least −50 mmHg to the biological tissue of the wound.

Further disclosed herein is a kit for treating or preventing localized fungal infection in a wound of a human patient. The kit may include at least one antifungal agent, a treatment delivery system and/or irrigation device configured to locally irrigate (instill and aspirate) a series of doses of the at least one antifungal agent, and instructions for administration. The instructions may include instructions for administering the at least one dose of the at least one antifungal agent to provide the patient continuous antifungal therapy for at least a 24-hour period. It is further contemplated that the instructions may include steps for administering the at least one antifungal agent and/or the at least one antibiotic according to any of the methods disclosed herein. In some embodiments, the at least one antifungal agent in the kit may include at least one antifungal agent and at least one additional antimicrobial agent, and in some embodiments, the at least one antimicrobial agent in the kit may include at least one antibiotic and/or at least one pain management agent (e.g. anesthetic or analgesic agent).

Also provided herein, in some aspects, is a kit for treating a localized infection in a human patient, the kit comprising: i) at least one antiseptic agent; ii) a treatment delivery system and/or irrigation device configured to locally irrigate the localized infection with at least one dose of the at least one antiseptic agent; and iii) instructions for administering the at least one antiseptic agent. In some aspects, the antiseptic agent comprises hypochlorous acid, sodium hypochlorite, citric acid, acetic acid, polyhexamethylene biguanide, chlorhexidine glutonate, povidone iodine, copper sulfate/potassium iodide, or taurolidine. In other aspects, the antiseptic agent comprises an antifungal agent. In some examples, the kit may further include one or more antibiotics. For example, the one or more antibiotics may be vancomycin, daptomycin, tobramycin, or any other antibiotic disclosed herein.

In some examples, the at least one antifungal agent and/or the at least one antibiotic may be provided in a vial or other container in an undiluted form. The end user may then combine the at least one antifungal agent and/or the at least one antibiotic with saline or other biocompatible fluid in one or more fluid reservoirs at the desired concentration once the antifungal agent(s) and/or antibiotic(s) are ready to administer. In other examples, the antifungal agent(s) and/or the antibiotic(s) may be provided in saline and stored in fluid reservoirs, such as a bag. In various embodiments, the kit may include the at least one antifungal agent and the one or more antibiotics and any combination of the treatment delivery system, irrigation device, fluid reservoirs, any other components needed for the treatment delivery system or irrigation device to function properly, and instructions for administering the antifungal agent(s) and antibiotic(s). In some instances, the kit may be provided separately to the end user such that separate elements of the kit may arrive to the end user at different times and then may be assembled by the end user prior to administration to a patient. The at least one antifungal agent and/or the at least one antibiotic may be provided in a solution with saline. The solution may further include one or more buffering agents. For example, the solution may include sodium bicarbonate. In other examples, the solution may further include dextrose or other excipients needed to effectively store and administer the antifungal agent and/or the antibiotic or pain management agent.

In some embodiments, the at least one antifungal agent may be fluconazole and the at least one antibiotic may be vancomycin, daptomycin, and/or tobramycin. The treatment delivery system and/or irrigation device is configured to locally administer, via irrigation, a daily dose of the at least one antifungal agent and/or the at least one antibiotic to the wound. The treatment delivery system and/or irrigation device may not include a sponge, which then requires intervention by a user to remove the sponge after a period of time. The treatment delivery system and/or irrigation device may be configured with multiple control features to complete administration from two or more sources of therapeutic agents for a single day without interruption or input from the patient or other user. In some examples, the multiple control features comprise two or more instillation pumps or two or more control valves, each operated independently by the treatment delivery system.

The treatment delivery system and/or irrigation device may further comprise local irrigation components to be applied on or in the wound to be treated. In some examples, the local irrigation components may comprise perforated instillation tubing, implantable fenestrated devices, drains, dressings, sponges, cannulas, or aspiration tubing. In some examples, the irrigation apparatus, including local irrigation components, is configured to not be removed from the patient until the administering is complete. The treatment delivery system and/or irrigation device includes local irrigation components which remain in place for up to 10 days. For example, the treatment delivery system and/or irrigation device is configured to be applied at the localized site of fungal infection (wound) for greater than 48 hours, at least 3 days, at least 4 days, at least 5 days, at least 6 days, at least 7 days, or at least 10 days. In other examples, the irrigation apparatus is configured to be applied at the localized site of fungal infection for 24 hours, less than 48 hours, less than 3 days, less than 4 days, less than 5 days, less than 6 days, less than 7 days, or less than 10 days.

The treatment delivery system and/or irrigation device may be configured to administer more than one agent (antifungal agent and/or other therapeutic agent) from more than one source without interruption or intervention by the user. In an example, the irrigation apparatus is configured to locally administer in a 24-hour period a total dose of fluconazole that equals or exceeds 200 mg, a total dose of tobramycin that equals or exceeds 50 mg and a total dose of vancomycin that equals or exceeds 2,000 mg without interruption or human intervention.

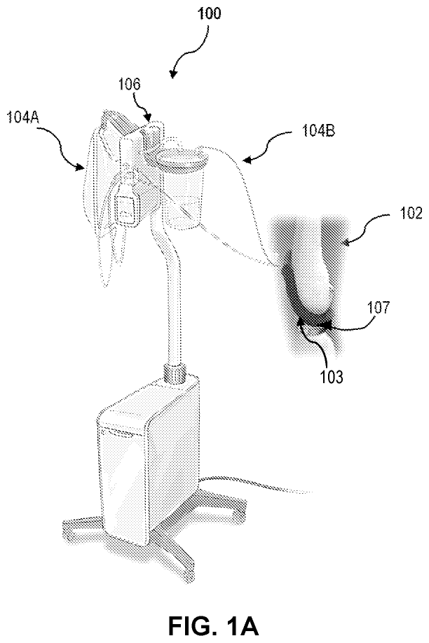

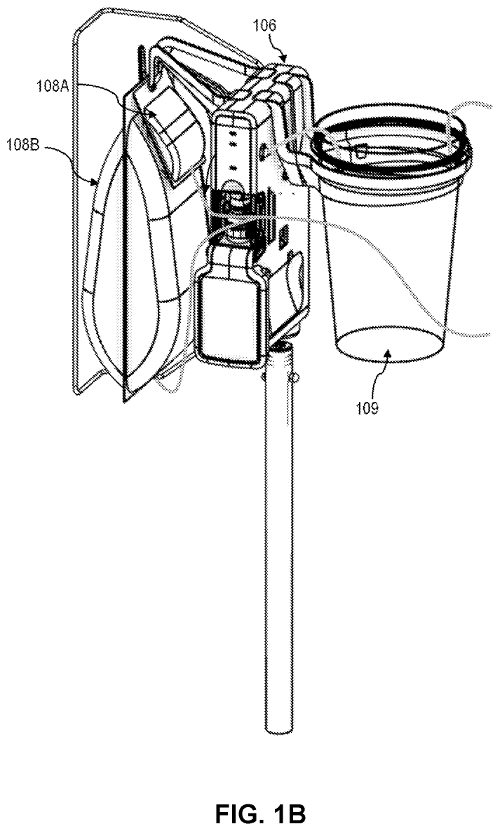

FIGS. 1A-1B show an example of a treatment delivery system 100 and irrigation device 107 that can provide controlled delivery of at least one antifungal agent into a treatment site 102 (e.g., infection in a periprosthetic space, bone segment, or wound area). All or portions of the treatment delivery system 100 and the irrigation device 107 can be included in the kit. In general, the treatment delivery system 100 can include a (portable) disposable device for enabling adjustment of a distance between the treatment delivery system 100 and a treated site 102 of a patient, such as a patient suffering from an infection treatable with fluid irrigation. In the example illustrated in FIG. 1A, the treatment delivery system 100 provides irrigation to a treatment site 102 including an irrigable volume (e.g., joint space or bone segment). The system 100 can include a fluid delivery system 104A, a fluid collection system 104B, and a control system 106. The fluid delivery system 104A can include one or more fluid reservoirs 108A, B. In some examples, the one or more fluid reservoirs 108A,B can contain the at least one antifungal agent and/or one or more antibiotics provided by the kit. For example, fluid reservoir 108A can contain an antifungal agent such as fluconazole, isavuconazole, posaconazole, or voriconazole in a saline solution and fluid reservoir 108B can contain an antibiotic such as vancomycin, daptomycin, or tobramycin in a saline solution. In other examples, fluid reservoir 108B can contain a second antifungal agent. The fluid collection system 104B can include one or more collection fluid canisters 109.

In some examples, the control system 106 can control fluid flow from the fluid reservoirs 108A,B towards the treatment site 102 and/or draw fluid away from the treatment site 102 toward the fluid collection system 104B. For example, the control system 106 can include one or more of a vacuum pressure source, pump, or any other means for moving fluid through the system 100. In some examples, one means for moving the fluid may be used for both instilling and aspirating fluid from the treatment site 102. In other examples, one means may be used for instillation and another means may be used for aspiration. The system can be configured to deliver the fluid transcutaneously to irrigation devices deployed within treated area (e.g., infected tissue and/or infected joints, such as hip, knee, shoulder, wrist, ankle). Although not shown, one or more other medical devices may be assisting and working in coordination and/or parallel with the treatment delivery system 100 to provide treatment for the patient (including the treatment site 102).

In an example, the irrigation device 107 can be configured to be fluidly coupled to the treatment delivery system 100 as shown in FIG. 1A. The treatment delivery system 100 can also be coupled to a syringe or another manual component for delivery and withdrawal of fluid from the treatment site. In some aspects, the irrigation device 107 may have a porous component 103. The porous component 103 of the irrigation device 107 may be configured to fill the space and/or opening of the treatment site and allow for fluid exiting the tubing to flow through the porous component 103 into the treatment site 102.

The treatment system 100 can be configured to fluidly couple with the irrigation device 107 that can provide controlled delivery of fluids (e.g., antibiotics, saline, local anesthetics) into the treatment site 102 (e.g., an infected articulating joint, such as knee or hip) during, for example, I&D, DAIR, Double DAIR, or another procedure as described elsewhere herein or other type of healing including wound and perioperative healing.

Within a context example, the treatment delivery system 100 can be used for delivering at least one antifungal agent and/or one or more antibiotics to infected joints (e.g., hip, knee, shoulder, wrist, ankle) or any type of biological tissue that can be treated using controlled fluid irrigation. Examples of treatments include periprosthetic joint infection treatment protocols, such as debridement, antibiotics and implant retention (DAIR) and exchange arthroplasty, irrigation of surgical incisions and traumatic wounds or irrigation for osteomyelitis and septic arthritis. Although the application may be described in the context of a particular treatment site (e.g., infected joints) and connection with that site, it should be appreciated that other treatment sites are considered and the way in which the system connects with those various sites may vary.

Again with respect to FIGS. 1A-1B, the fluid delivery system 104A includes two or more fluid reservoir(s) 108A, 108B.

Each of the fluid reservoirs 108A, 108B can have a particular volume and store a particular fluid type that is delivered during a respective treatment phase. The contents of the fluid reservoirs may be switched such that an antifungal agent may be stored in a first fluid reservoir 108B or a second fluid reservoir 108A and one or more antibiotics may be stored in a second fluid reservoir 108A or a first fluid reservoir 108B. The fluid reservoirs may also contain combinations of at least one antifungal agent and antibiotics in some instances. The fluid reservoirs may be provided as part of the kit or may be provided separately and combined with the one or more antifungal agents and one or more antibiotics of the kit prior to use.

For example, the first fluid reservoir 108A can contain or store a first fluid that can include at least one antifungal agent, such as fluconazole, isavuconazole, posaconazole, or voriconazole. The first fluid can be delivered from first fluid reservoir 108A to the treatment site 102 according to a respective fluid delivery protocol. The fluid delivery protocol can be included within the instructions of a kit. The protocol can define a concentration of at least one antifungal agent, a volume of the antifungal agent fluid to be delivered, a duration of antifungal agent fluid delivery, and pre-delivery vacuum that can be performed for a set vacuum time period (e.g., approximately 30 minutes) at a set pressure (e.g., −125 mmHg). The total volume of the antifungal agent fluid delivered can vary depending on the length of the time period as well as the total time for first fluid delivery, soak, and removal. The delivery of antifungal agent fluid is followed by a soaking protocol to enable the treatment site 102 to soak the delivered fluid. The duration of the antifungal agent fluid soaking protocol can be between 15 and 45 minutes, such as approximately 30 minutes. The antifungal agent fluid can be removed from the treatment site 102, by the fluid collection system 104B. The duration of the second fluid removal from the treatment site 102 can be approximately 30 minutes.

The second fluid reservoir 108B can store a second fluid that can include an antibiotic, such as tobramycin, vancomycin, or daptomycin or a second antifungal agent, such as fluconazole, isavuconazole, posaconazole, or voriconazole. The total volume of the second fluid delivered can vary depending on the length of the time period as well as the total time for first fluid delivery, soak, and removal. The delivery of the second fluid is followed by a soaking protocol to enable the treatment site 102 to soak the delivered fluid. The duration of the second fluid soaking protocol can be between 15 and 45 minutes, such as approximately 30 minutes. The second fluid can be removed from the treatment site 102, by the fluid collection system 104B. The duration of the antibiotic fluid removal from the treatment site 102 can be approximately 30 minutes.

The fluid collection system 104B can direct fluid from the treatment site 102 to a collection fluid canister 109. In some examples, the control system 106 can generate a vacuum in the collection fluid canister 109 to remove the fluid from the treatment site 102 into the collection fluid canister 109 through the vacuum line 113A. For example, a pump can be activated by the control system 106 to create a vacuum within at least the collection fluid canister 109. In other examples, vacuum pressure sources, syringes, gravity, pumps, or any other mechanism for moving fluid in and out of the treatment site 102 are contemplated. The collection fluid canister 109 can include a single-use canister with a known volume (e.g., 2000 mL). The collection fluid canister 109 collects antibiotic solution or other fluids (e.g., wound exudate) accumulated during vacuum-induced drainage of fluid from the treatment site 102. The material of the canister 109 is preferably translucent or transparent in order for a user to assess the level of the contents within the canister 109 during use. The material of the canister 109 is also preferably capable of retaining a vacuum without deforming or restricting flow at maximum vacuum level.

### Definitions

In various implementations, description is made with reference to the figures. However, certain implementations may be practiced without one or more of these specific details, or in combination with other known methods and configurations. In the description, many specific details are set forth, such as specific doses, fixed dose combinations and concentrations, to provide a thorough understanding of the invention. In other instances, specific doses, fixed dose combinations and concentrations have not been described in detail to not unnecessarily obscure the description. Reference throughout this specification to “dose,” “fixed dose ratio,” “concentration” or the like, means that a particular method, or characteristic described is included in at least one novel method of treatment. Thus, the appearance of the phrase “one method of treatment,” or the like, in various places throughout this specification is not necessarily referring to the same method of treatment. Furthermore, the particular antibiotics, doses, fixed dose combinations, ratios and concentrations may be combined in any suitable manner in one or more novel method of treatment.

As used herein, the term “antifungal” refers to an agent capable of inhibiting the growth of, or destroying, fungal cells such as, for example hyphal cells, on living tissue, surfaces, or wounds, thereby reducing or preventing fungal infections. Antifungal agents may include, by way of non-limiting example, agents that also possess activity against non-fungal microorganisms, including antiseptic agents. Accordingly, “antiseptic agent” as used herein is intended to refer to a class or subset of antifungal agents that exhibit broad-spectrum activity against bacteria, viruses, or other microbes in addition to fungi. Unless otherwise specified, the methods, formulations, and dosing described here for antifungal agents are equally applicable to antiseptic agents.

Antifungal agents, within the scope of this disclosure, may comprise antiseptic agents, antibacterial agents, and antiviral agents, provided such agents exhibit activity against fungal pathogens. Where the specification refers to the use or administration of “antiseptic agents,” it is to be understood that such references contemplate their inclusion within the broader category of “antifungal agents,” unless the context indicates otherwise. In certain embodiments, the terms “antifungal agent” and “antiseptic agent” may be used interchangeably.

Where a range of values is provided, each value between the upper and lower ends of the range is specifically contemplated and described herein.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value and may include the specified value. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL” The endpoint may also be based on the variability allowed by an appropriate regulatory body, such as the FDA, USP, etc.

As used herein, the term “substantially” means a value a person of ordinary skill in the art would consider significantly more than a specified value. In some aspects, substantially means at least 25% more than the specified value, or at least 125% of the specified value.

As used herein, the terms “safe” and “recommended” means recommended by a medical authority, such as a manufacturer, the FDA or other similar regulatory body. In some examples, the maximum dose or serum level may be based on peer reviewed medical literature, for example Rybak et al., “Therapeutic Monitoring of Vancomycin in Adult Patients: A Consensus Review of the American Society of Health-System Pharmacists, the Infections Diseases Society of America, and the Society of Infectious Diseases Pharmacists” ASHP Therapeutic Position Statements, p. 685-700, 2009, or recommended by a professional medical organization or association. In additional examples, a maximum recommended daily systemic dose and/or a maximum safe serum concentration may be provided in the label or instructions for use of the particular antiseptic, antifungal agent, or antibiotic.

As used herein, the terms “maximum recommended daily dose,” “maximum recommended daily systemic dose” and “maximum recommended systemic daily dose” may be used interchangeably. In some examples, the systemic daily dose may be a daily dose administered by IV.

While this specification contains many specifics, these should not be construed as limitations on the scope of what is claimed or of what may be claimed, but rather as descriptions of features specific to particular methods. Certain methods that are described in this specification may also be implemented in combination in a single method. Conversely, various features that are described in the context of a single method may also be implemented individually, in multiple methods separately or in any suitable sub-combination of novel methods. Moreover, although methods may be described herein as including certain combinations of antibiotics, doses or concentrations and even initially claimed as such, one or more features from such a claimed combination may in some cases be excised from the combination, and the claimed combination may be directed to a sub-combination or a variation of a sub-combination. Similarly, while administration of more than one antibiotic sequentially is described in the specification in a particular order or for particular durations, this should not be understood as requiring that such methods be performed in the particular sequential order or for the particular durations, or that all elements of the sequence be performed, to achieve desirable results. Only a few examples and methods are disclosed. Variations, modifications and enhancements to the described methods and other methods may be made based on what is disclosed.

In the descriptions above and in the claims, phrases such as “at least one” or “one or more” may occur followed by one or a conjunctive list of elements, antibiotics, combinations, doses or concentrations. The term “and/or” may also occur in a list of two or more such elements. Unless otherwise implicitly or explicitly contradicted by the context in which it is used, such a phrase is intended to mean any of the listed elements individually or any of the recited elements in combination with any of the other recited elements. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on” above and in the claims is intended to mean, “based at least in part on,” such that an unrecited feature or element is also permissible.

As used herein, “comprises,” “comprising,” “containing,” and “having” and the like may have the meaning ascribed to them in U.S. Patent Law and may mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, which do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open-ended term, like “comprising” or “including,” it is understood that direct support should be afforded also to “consisting essentially of” language as well as “consisting of” language as if stated explicitly and vice versa.

As used herein, first, second, third, etc. are used to characterize and distinguish various elements of the invention. Use of numerical terms may be used to distinguish one drug from another. Use of such numerical terms does not imply a sequence or order unless clearly indicated by the context. Such numerical references may be used interchangeably without departing from the teaching of the embodiments and variations herein.

The inventions disclosed herein may be packaged together in a single package of multiple therapeutic agents or in multiple packages of individual or combinations of therapeutic agents for use together, and such use may be administered concurrently, serially, cyclically or sequentially (in a particular sequence which may include repeat use of one or more individual or combination of therapeutic agents) per specific instructions. All methods described herein may be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claims. No language in the specification should be construed as indicating any non-claimed element essential to the practice of the invention.

### EMBODIMENTS

Embodiment 1: A method of treating or preventing a fungal infection, the method comprising: locally administering to a human patient a plurality of doses of at least one antifungal agent in solution by irrigating the at least one antifungal agent at a wound, wherein the administration provides the patient continuous localized antifungal therapy for at least a 24-hour period.

Embodiment 2: The method of embodiment 1, wherein the administering provides a serum concentration of the at least one antifungal agent less than or equal to a maximum safe level for serum concentration of the at least one antifungal agent in the human patient.

Embodiment 3: The method of embodiment 1, wherein the administration provides the patient continuous localized antifungal therapy for at least a 48-hour period.

Embodiment 4: The method of embodiment 3, wherein the administration provides the patient continuous localized antifungal therapy for at least 3 days, 4 days, 5 days, 6 days, at least 7 days, or at least 10 days.

Embodiment 5: The method of embodiment 1, wherein the irrigating comprises alternating instillation and aspiration cycles.

Embodiment 6: The method of embodiment 5, wherein the instillation and aspiration cycles are sequential.

Embodiment 7: The method of embodiment 5, wherein the instillation and aspiration cycles are staggered.

Embodiment 8: The method of embodiment 5, wherein an irrigating apparatus is configured to control the irrigating automatically.

Embodiment 9: The method of embodiment 5, wherein the irrigating comprises soaking the localized site of fungal infection with the at least one antifungal agent for a time interval between each alternating instillation and aspiration cycle.

Embodiment 10: The method of embodiment 9, wherein a total daily dose of the antifungal agent that is locally administered by irrigation is less than or equal to established FDA approved intravenous doses of the antifungal agent.

Embodiment 11: The method of embodiment 9, wherein the time interval is for about 10 minutes to about 180 minutes.

Embodiment 12: The method of embodiment 9, wherein the time interval is for about 30 minutes to about 120 minutes.

Embodiment 13: The method of embodiment 9, wherein the aspiration cycle is less than 60 minutes.

Embodiment 14: The method of embodiment 1, wherein the irrigating is performed at least twice in a 24-hour period.

Embodiment 15: The method of embodiment 1, wherein the irrigating is performed at least 4 times in a 24-hour period.

Embodiment 16: The method of embodiment 1, wherein the irrigating is performed at least 8 times in a 24-hour period.

Embodiment 17: The method of embodiment 1, wherein the irrigating is performed at least 16 times in a 24-hour period.

Embodiment 18: The method of embodiment 1, wherein the irrigating is performed continuously over a 24-hour period.

Embodiment 19: The method of embodiment 1, wherein the at least one antifungal agent is an azole.

Embodiment 20: The method of embodiment 19, wherein the azole is selected from the group consisting of fluconazole, isavuconazole, posaconazole, voriconazole, and combinations thereof.

Embodiment 21: The method of embodiment 1, wherein the amount of the antifungal agent locally administered in the 24-hour period is at least 100 mg.

Embodiment 22: The method of embodiment 21, wherein the locally administered dose of the antifungal agent is at a concentration of at least 80 mcg/mL and the administering provides a serum concentration of fluconazole of less than 5 mcg/mL in the human patient.

Embodiment 23: The method of embodiment 21, wherein the amount of the antifungal agent locally administered in the 24-hour period is at least 200 mg, 400 mg, 800 mg, 1,200 mg, or 1,600 mg.

Embodiment 24: The method of embodiment 21, wherein the locally administered dose of the antifungal agent is at a concentration of at least 600 mcg/mL and the administering provides a serum concentration of the antifungal agent of less than 5 mcg/mL in the human patient.

Embodiment 25: The method of embodiment 21, wherein the amount of the antifungal agent locally administered in the 24-hour period is at least 1,200 mg.

Embodiment 26: The method of embodiment 21, wherein the amount of the antifungal agent locally administered in the 24-hour period exceeds the recommended daily dose of the antifungal agent administered intravenously or systemically to an adult human patient for a 24-hour period.

Embodiment 27: The method of embodiment 21, wherein the amount of the antifungal agent locally administered is 100 to 3,000 mg in the 24-hour period, and the administering provides a serum concentration of the antifungal agent of less than 5 mcg/mL in the human patient.

Embodiment 28: The method of embodiment 21, wherein the amount of the antifungal agent locally administered in the 24-hour period is at least 500 mg to 1,500 mg.

Embodiment 29: The method of embodiment 21, wherein the serum concentration of the antifungal agent is less than 2.5 mcg/mL in the human patient.

Embodiment 30: The method of embodiment 29, wherein the serum concentration of the antifungal agent is less than 1.25 mcg/mL in the human patient.

Embodiment 31: The method of embodiment 1, wherein the antifungal agent is fluconazole or voriconazole.

Embodiment 32: The method of embodiment 31, wherein the amount of fluconazole or voriconazole locally administered in the 24-hour period is at least 100 mg.

Embodiment 33: The method of embodiment 32, wherein the locally administered dose of fluconazole or voriconazole is at a concentration of at least 80 mcg/mL and the administering provides a serum concentration of fluconazole or voriconazole of less than 5 mcg/mL in the human patient.

Embodiment 34: The method of embodiment 32, wherein the amount of fluconazole or voriconazole locally administered in the 24-hour period is at least 200 mg, 400 mg, 800 mg, 1,200 mg, or 1,600 mg.

Embodiment 35: The method of embodiment 32, wherein the locally administered dose of fluconazole or voriconazole is at a concentration of at least 600 mcg/mL and the administering provides a serum concentration of fluconazole or voriconazole of less than 5 mcg/mL in the human patient.

Embodiment 36: The method of embodiment 32, wherein the amount of locally administered fluconazole or voriconazole in the 24-hour period is at least 1,200 mg.

Embodiment 37: The method of embodiment 32, wherein the amount of locally administered fluconazole or voriconazole in the 24-hour period exceeds the recommended daily dose of fluconazole or voriconazole administered intravenously or systemically to an adult human patient for a 24-hour period.

Embodiment 38: The method of embodiment 32, wherein the amount of locally administered fluconazole or voriconazole is 100 to 3,000 mg in the 24-hour period, and the administering provides a serum concentration of fluconazole or voriconazole of less than 5mcg/mL in the human patient.

Embodiment 39: The method of embodiment 32, wherein the amount of locally administered fluconazole or voriconazole in the 24-hour period is at least 500 mg to 1,500 mg.

Embodiment 40: The method of embodiment 32, wherein the serum concentration of fluconazole or voriconazole is less than 2.5 mcg/mL in the human patient.

Embodiment 41: The method of embodiment 40, wherein the serum concentration of fluconazole or voriconazole is less than 1.25 mcg/mL in the human patient.

Embodiment 42: The method of embodiment 1, wherein the at least one antifungal agent is amphotericin B or lipid formulations thereof.

Embodiment 43: The method of embodiment 1, wherein the at least one antifungal agent is an echinocandin.

Embodiment 44: The method of embodiment 43, wherein the echinocandin is selected from the group consisting of anidulafungin, caspofungin, micafungin, and combinations thereof.

Embodiment 45: The method of embodiment 44, wherein the amount of locally administered echinocandin is at least 50 mg in the 24-hour period.

Embodiment 46: The method of embodiment 1, wherein the wound is a topical wound.

Embodiment 47: The method of embodiment 46, wherein the topical wound is a diabetic ulcer.

Embodiment 48: The method of embodiment 1, wherein the wound is a traumatic wound.

Embodiment 49: The method of embodiment 48, wherein the traumatic wound includes an implanted medical device.

Embodiment 50: The method of embodiment 1, wherein the wound is a surgical wound.

Embodiment 51: The method of embodiment 50, wherein the surgical wound is a subcutaneous surgical wound.

Embodiment 52: The method of embodiment 51, wherein the subcutaneous surgical wound includes an implanted medical device.

Embodiment 53: The method of embodiment 52, wherein the implanted medical device is an orthopedic implant.

Embodiment 54: The method of embodiment 53, wherein the orthopedic implant is a joint replacement prosthesis.

Embodiment 55: The method of embodiment 52, wherein therapy time for the implanted medical device is reduced due to the locally administering of the at least one antifungal agent.

Embodiment 56: The method of embodiment 55, wherein the therapy time is reduced by at least 1 to 10 days as compared to a therapy time for the implanted medical device without locally administering at least one antifungal agent.

Embodiment 57: The method of embodiment 1, wherein the wound is a site of localized fungal infection.

Embodiment 58: The method of embodiment 57, wherein the localized antifungal therapy is provided up to at least 15 cm from the wound.

Embodiment 59: The method of embodiment 1, wherein the irrigation occurs within a perimeter of a topical wound or a subcutaneous wound.

Embodiment 60: The method of embodiment 1, further comprising locally administering at least one additional therapeutic agent for treatment of a condition other than fungal infection.

Embodiment 61: The method of embodiment 60, wherein the at least one additional therapeutic agent is at least one antimicrobial agent.

Embodiment 62: The method of embodiment 61, wherein the at least one antimicrobial agent is two or more antibiotics.

Embodiment 63: The method of embodiment 62, wherein the two or more antibiotics comprise vancomycin and tobramycin.

Embodiment 64: The method of embodiment 62, wherein the two or more antibiotics are sequentially administered.

Embodiment 65: The method of embodiment 64, wherein the at least one antifungal agent is fluconazole.

Embodiment 66: The method of embodiment 65, wherein the fluconazole and vancomycin are administered concurrently, and the tobramycin is administered sequentially to the concurrently administered fluconazole and vancomycin.

Embodiment 67: The method of embodiment 65, wherein the serum concentrations of each of the fluconazole, the vancomycin and the tobramycin remain below the recommended safe serum concentrations of each of the fluconazole, the vancomycin and the tobramycin throughout the time-period of administration.

Embodiment 68: The method of embodiment 61, wherein the at least one antifungal agent is amphotericin B.

Embodiment 69: The method of embodiment 68, wherein the amphotericin B is administered sequentially with the at least one antimicrobial agent.

Embodiment 70: The method of embodiment 69, wherein the at least one antimicrobial agent is tobramycin.

Embodiment 71: The method of embodiment 61, wherein the at least one antifungal agent is administered sequentially with the at least one antimicrobial agent.

Embodiment 72: The method of embodiment 61, wherein the at least one antifungal agent is administered concurrently with the at least one antimicrobial agent.

Embodiment 73: The method of embodiment 61, wherein there is no drug-drug interaction between the at least one antifungal agent and the at least one antimicrobial agent.

Embodiment 74: The method of embodiment 1, wherein the local administration is provided for 1 to 10 days.

Embodiment 75: The method of embodiment 1, wherein the at least one antifungal agent is in a solution comprising saline.

Embodiment 76: The method of embodiment 75, wherein the solution further comprises dextrose.

Embodiment 77: The method of embodiment 1, wherein the concentration of the at least one antifungal agent exceeds a maximum recommended concentration of the at least one antifungal agent.

Embodiment 78: The method of embodiment 77, wherein the local administration comprises irrigating the localized wound by soaking it with the at least one antifungal agent and aspirating the at least one antifungal agent after a period of time.

Embodiment 79: The method of embodiment 1, wherein the patient has reduced side effects after the local administration as compared to administering the at least one antifungal agent topically.

Embodiment 80: The method of embodiment 79, wherein the patient has reduced irritation, itching, or systemic inflammatory response.

Embodiment 81: The method of embodiment 1, wherein the patient has immunosuppression, cancer, renal insufficiency, or a combination thereof.

Embodiment 82: The method of embodiment 81, wherein the immunosuppression in the human patient is due to an auto-immune disorder of the human patient or due to immunosuppressing medication administered to the human patient.

Embodiment 83: The method of embodiment 1, wherein persistent infection or reinfection of the fungal infection is prevented in the patient.

Embodiment 84: The method of embodiment 1, wherein there is no risk or substantially no risk of ototoxicity to the human patient.

Embodiment 85: The method of embodiment 1, wherein there is no risk or substantially no risk of nephrotoxicity to the human patient.

Embodiment 86: A kit for managing localized antifungal therapy in a human patient, the kit comprising: at least one antifungal agent; a treatment delivery system and/or irrigation device configured to locally irrigate a localized site of fungal infection with at least one dose of the at least one antifungal agent in solution; and instructions for administering the at least one dose of the at least one antifungal agent to provide the patient continuous localized antifungal therapy for at least a 24-hour period.

Embodiment 87: The kit of embodiment 86, wherein the at least one antifungal agent is fluconazole.

Embodiment 88: The kit of embodiment 87, wherein the irrigation device is configured to locally administer in the 24-hour period a total amount of fluconazole that equals or exceeds 100 mg.

Embodiment 89: The kit of embodiment 86, wherein the kit further comprises at least one antimicrobial agent.

Embodiment 90: The kit of embodiment 89, wherein the at least one antifungal agent is provided as a solution in a first independent source and a first antimicrobial agent is provided in a second independent source.

Embodiment 91: The kit of embodiment 89, wherein a second antimicrobial agent is provided in the first independent source with the at least one antifungal agent.

Embodiment 92: The kit of embodiment 89, wherein an anesthetic or analgesic is provided in the second independent source with the first antimicrobial agent.

Embodiment 93: The kit of embodiment 86, wherein the irrigation device is configured to not be removed from the patient until the administering is complete.

Embodiment 94: The kit of embodiment 93, wherein the irrigation device is configured to be applied at the localized site of fungal infection for greater than 48 hours.

Embodiment 95: The kit of embodiment 93, wherein the irrigation device is configured to be applied at the localized site of fungal infection for at least 3 days, at least 4 days, at least 5 days, at least 6 days, at least 7 days, or at least 10 days.

Embodiment 96: The kit of embodiment 86, wherein the at least one antifungal agent is in a solution comprising saline.

Embodiment 97: The kit of embodiment 86, wherein the irrigation device includes local irrigation components which remain in place for up to 10 days.

Embodiment 98: A method of treating or preventing a localized fungal infection in a wound of a human patient, the method comprising: locally administering by irrigation to the human patient repeated doses of at least two therapeutic agents from separate sources without interruption to change sources, wherein one of the two or more therapeutic agents is at least one antifungal agent.

Embodiment 99: The method of embodiment 98, wherein the two or more therapeutic agents are administered sequentially.

Embodiment 100: The method of embodiment 98, wherein the two or more therapeutic agents are administered concurrently.

Embodiment 101: A method of treating or preventing a localized fungal infection in a human patient in need thereof, the method comprising: locally administering by irrigation to the human patient repeated doses of two therapeutic agents from separate sources without interruption to change sources, wherein one of the two or more therapeutic agents is at least one antifungal agent and at least one antimicrobial agent.

Embodiment 102: The method of embodiment 101, wherein the at least one antimicrobial agent is at least one antibiotic.

Embodiment 103: The method of embodiment 102, wherein the at least one antimicrobial agent is at least two antibiotics.

Embodiment 104: The method of embodiment 103, wherein the at least one antifungal agent is fluconazole and the at least two antibiotics are vancomycin and tobramycin.

Embodiment 105: The method of embodiment 104, wherein the fluconazole and vancomycin are administered concurrently, and the tobramycin is administered sequentially with the concurrent lidocaine and vancomycin.

Embodiment 106: The method of embodiment 105, wherein the fluconazole and vancomycin are administered from a common source.

Embodiment 107: A method of treating or preventing a localized fungal infection in a human patient in need thereof, the method comprising: locally administering by irrigation to the human patient at least one antifungal agent and at least two antimicrobial agents.

Embodiment 108: The method of embodiment 107, wherein the at least one antifungal agent is fluconazole and the at least two antimicrobial agents are vancomycin and tobramycin.

Embodiment 109: A method of treating or preventing a local infection in a human patient at a localized site, the method comprising locally administering by irrigation to the localized site of the human patient a plurality of doses of: at least one antiseptic agent in solution; and at least one antibiotic in solution.

Embodiment 110: The method of embodiment 109, wherein the antiseptic agent comprises hypochlorous acid, sodium hypochlorite, citric acid, acetic acid, polyhexamethylene biguanide, chlorhexidine glutonate, povidone iodine, copper sulfate/potassium iodide, or taurolidine.

Embodiment 111: The method of embodiment 109, wherein at least one antibiotic comprises vancomycin, daptomycin, and/or tobramycin.

Embodiment 112: The method of embodiment 109, wherein the local infection comprises a bacterial pathogen, a fungal pathogen, or a polymicrobial pathogen.

Embodiment 113: The method of embodiment 109, wherein irrigating the localized site comprises instillation of the at least one antiseptic agent in solution followed by a dwell period followed by aspiration of the at least one antiseptic agent in solution to remove the at least one antiseptic agent in solution.

Embodiment 114: The method of embodiment 113, wherein about 50 mL of the at least one antiseptic agent in solution is instilled with about a 5-minute dwell period followed by about a 5-minute aspiration.

Embodiment 115: The method of embodiment 109, wherein the least one antiseptic agent in solution is locally administered by irrigation prior to the at least one antibiotic in solution.

Embodiment 116: A method of treating or preventing a local infection in a wound, the method comprising: locally administering to a human patient a plurality of doses of at least one antiseptic agent in solution by irrigating the wound, wherein the locally administering is performed at least once in a 24-hour period.

Embodiment 117: The method of embodiment 116, wherein the antiseptic agent comprises hypochlorous acid, sodium hypochlorite, citric acid, acetic acid, polyhexamethylene biguanide, chlorhexidine glutonate, povidone iodine, copper sulfate/potassium iodide, or taurolidine.

Embodiment 118: The method of embodiment 116, wherein the infection comprises a bacterial pathogen, a fungal pathogen or a polymicrobial pathogen.

Embodiment 119: The method of embodiment 116, wherein irrigating the wound with the at least one antiseptic agent in solution is performed prior to a daily local antibiotic irrigation.

Embodiment 120: The method of embodiment 119, wherein irrigating the wound comprises instillation of the at least one antiseptic agent in solution followed by a dwell period followed by aspiration of the at least one antiseptic agent in solution to remove the at least one antiseptic agent in solution.

Embodiment 121: The method of embodiment 120, wherein about 50 mL of the at least one antiseptic agent in solution is instilled with about a 5-minute dwell period followed by about a 5-minute aspiration.

Embodiment 122: The method of embodiment 119, wherein a sterile fluid is irrigated after the at least one antiseptic agent in solution to remove or otherwise dilute the at least one antiseptic agent.

Embodiment 123: The method of embodiment 122 wherein the sterile fluid comprises normal saline.

Embodiment 124: The method of embodiment 116, wherein irrigating the wound with the at least one antiseptic agent in solution is accomplished by simultaneously instilling and aspirating the at least one antiseptic agent in solution.

Embodiment 125: The method of embodiment 124, wherein at least about 100 mL, at least about 250 mL, at least about 500 mL, at least about 1000 mL or at least about 2000 mL of the at least one antiseptic agent in solution is irrigated.

Embodiment 126: The method of embodiment 125, wherein the at least one antiseptic agent in solution is irrigated in less than about 15 minutes.

Embodiment 127: A kit for treating a localized infection in a human patient, the kit comprising: at least one antiseptic agent; a treatment delivery system and/or irrigation device configured to locally irrigate the localized infection with at least one dose of the at least one antiseptic agent; and instructions for administering the at least one antiseptic agent.

What is claimed is:

1. A method of treating or preventing a local infection in a human patient at a localized site, the method comprising locally administering by irrigation to the localized site of the human patient a plurality of doses of:
i) at least one antiseptic agent in solution; and
ii) at least one antibiotic in solution.

2. The method of claim 1, wherein the antiseptic agent comprises hypochlorous acid, sodium hypochlorite, citric acid, acetic acid, polyhexamethylene biguanide, chlorhexidine glutonate, povidone iodine, copper sulfate/potassium iodide, or taurolidine.

3. The method of claim 1, wherein at least one antibiotic comprises vancomycin, daptomycin, and/or tobramycin.

4. The method of claim 1, wherein the local infection comprises a bacterial pathogen, a fungal pathogen, or a polymicrobial pathogen.

5. The method of claim 1, wherein irrigating the localized site comprises instillation of the at least one antiseptic agent in solution followed by a dwell period followed by aspiration of the at least one antiseptic agent in solution to remove the at least one antiseptic agent in solution.

6. The method of claim 5, wherein about 50 mL of the at least one antiseptic agent in solution is instilled with about a 5-minute dwell period followed by about a 5-minute aspiration.

7. The method of claim 1, wherein the least one antiseptic agent in solution is locally administered by irrigation prior to the at least one antibiotic in solution.

8. A method of treating or preventing a local infection in a wound, the method comprising: locally administering to a human patient a plurality of doses of at least one antiseptic agent in solution by irrigating the wound, wherein the locally administering is performed at least once in a 24-hour period.

9. The method of claim 8, wherein the antiseptic agent comprises hypochlorous acid, sodium hypochlorite, citric acid, acetic acid, polyhexamethylene biguanide, chlorhexidine glutonate, povidone iodine, copper sulfate/potassium iodide, or taurolidine.

10. The method of claim 8, wherein the infection comprises a bacterial pathogen, a fungal pathogen or a polymicrobial pathogen.

11. The method of claim 8, wherein irrigating the wound with the at least one antiseptic agent in solution is performed prior to a daily local antibiotic irrigation.

12. The method of claim 11, wherein irrigating the wound comprises instillation of the at least one antiseptic agent in solution followed by a dwell period followed by aspiration of the at least one antiseptic agent in solution to remove the at least one antiseptic agent in solution.

13. The method of claim 12, wherein about 50 mL of the at least one antiseptic agent in solution is instilled with about a 5-minute dwell period followed by about a 5-minute aspiration.

14. The method of claim 11, wherein a sterile fluid is irrigated after the at least one antiseptic agent in solution to remove or otherwise dilute the at least one antiseptic agent.

15. The method of claim 14 wherein the sterile fluid comprises normal saline.

16. The method of claim 8, wherein irrigating the wound with the at least one antiseptic agent in solution is accomplished by simultaneously instilling and aspirating the at least one antiseptic agent in solution.

17. The method of claim 16, wherein at least about 100 mL, at least about 250 mL, at least about 500 mL, at least about 1000 mL or at least about 2000 mL of the at least one antiseptic agent in solution is irrigated.

18. The method of claim 17, wherein the at least one antiseptic agent in solution is irrigated in less than about 15 minutes.

19. A kit for treating a localized infection in a human patient, the kit comprising:
i) at least one antiseptic agent;
ii) a treatment delivery system and/or irrigation device configured to locally irrigate the localized infection with at least one dose of the at least one antiseptic agent; and
iii) instructions for administering the at least one antiseptic agent.
